(19) United States
(12) Patent Application Publication
(10) Pub. No.: US 2026/0102150 A1
(43) Pub. Date: Apr. 16, 2026

(54) SOFT ANCHOR DEPLOYMENT SYSTEMS AND METHODS OF USE THEREOF

(71) Applicants: Smith & Nephew, Inc., Memphis, TN (US); Smith & Nephew Asia Pacific Pte. Limited, Singapore (SG)

(72) Inventor: Mark E. Housman, North Attleboro, MA (US)

(21) Appl. No.: 19/354,127

(22) Filed: Oct. 9, 2025

Related U.S. Application Data
(60) Provisional application No. 63/706,202, filed on Oct. 11, 2024.

(51) Int. Cl. A61B 17/04; A61F 2/08

(52) U.S. Cl. CPC A61B 17/0401; A61F 2/0811; A61B 2017/0409; A61B 2017/0464; A61B 2017/0496; A61F 2002/0817

(57) ABSTRACT

A soft anchor deployment system for tissue repair includes an insertion instrument having a shaft extending up to a distally facing surface. A tip member extends distally from the distal facing surface. A soft anchor is positioned on the tip member such that the tip member is disposed within and along a lumen of the soft anchor. A suture operatively couples to the soft anchor and to a tensioning actuator of the insertion instrument. Tension applied to the suture deploys the soft anchor from a first elongated state to a second expanded state. During deployment of the soft anchor, a distal end of the soft anchor slides along and over the tip member and the soft anchor is compressed against the distally facing surface such that the tip member extends distally from the distal end of the soft anchor.

### CROSS-REFERENCED TO RELATED APPLICATIONS

This application claims priority to and benefit of U.S. Provisional Application No. 63/706,202, filed on Oct. 11, 2024, entitled ALL-SUTURE ANCHOR SYSTEM, the entire contents of which are incorporated herein by reference for all purposes.

### FIELD

The present disclosure relates generally to soft-bodied suture anchors and associated deployment systems for tissue repair.

### BACKGROUND

Surgeons often use soft suture anchors during tissue repair because they allow for placement of anchors in areas of the body where only a small anchor can be used, and because use of such anchors can avoid placing plastic or metal within the body. Soft suture anchors typically contain a tubular or sheath-like body made of a soft, flexible material, and a suture interwoven throughout the body or otherwise coupled to the body. When the anchor is in an undeployed, insertable state, it is typically elongate such that a length of the body is greater than its width. Tension applied to the suture causes the body to expand radially and shorten axially, forming an interference fit within the surrounding tissue, such as a bone hole.

Inserting soft suture anchors into tissue generally requires rigid elements to prevent the anchors from folding or buckling during insertion. These rigid elements keep the soft anchor in its elongate, small diameter form, allowing for insertion into smaller bone or tissue holes. For some anchor systems, the soft anchors are housed within a tube on the anchor inserter, which acts as the rigid element keeping the soft anchor in its elongate form before tension is applied to the suture.

### SUMMARY

Insertion tubes that house soft anchors before deployment may be awkward to assemble and may also add bulk to the inserter distal end. Therefore, it would be desirable to provide a soft anchor deployment system for deploying and tensioning a soft suture anchor that has a minimal profile (i.e., small diameter) and is simple to assemble and actuate. Such systems may also provide a reduced cost system that requires less tissue removal and is less complicated to use than systems using a rigid outer tube.

This disclosure describes a soft anchor deployment system that provides an inserter with an inner shaft that extends within and along a lumen of a soft suture anchor. This inner shaft may place the soft suture anchor within or through a target tissue while maintaining the soft anchor in its elongate or undeployed configuration. During deployment, the soft suture anchor may slide along this inner shaft, which remains stationary. The inner shaft may be removed from the soft suture anchor post deployment. The inner shaft may include slots, holes or surfaces that provide space for the tensioning sutures to slide during deployment. The soft anchor may be deployed with a high degree of tensioning, creating 360-degree anchor expansion. The soft suture anchor may be exposed at a distal end of the inserter and avoid the need for an insertion outer tube.

Embodiments of the soft anchor deployment system for tissue repair of this disclosure may include one or more of the following, in any suitable combination.

In embodiments, the soft anchor deployment system of this disclosure includes an insertion instrument having a handle with a tensioning actuator at a proximal end and a shaft extending distally from the handle up to a distally facing surface. A tip member extends distally from the distal facing surface and has a first region. A soft anchor includes a tubular body defining a lumen. The tubular body is positioned on the tip member such that the tip member is disposed within and along the lumen. A suture operatively couples to the tubular body and to the tensioning actuator. Tension applied to the suture deploys the tubular body from a first elongated state to a second expanded state. During deployment, a distal end of the tubular body slides along and over the tip member and the tubular body is compressed against the distally facing surface such that the tip member extends distally from a distal end of the tubular body.

In further embodiments, the tip member has a second region configured to provide slidable clearance for passage of limbs of the suture within the shaft. In embodiments, the second region includes a tab having a wider portion defining flat opposing sides. A diameter of the wider portion is selected to match an inner diameter of the shaft. In embodiments, the flat opposing sides of the tab provide the slidable clearance for the passage of the limbs of the suture between the tab and the shaft. In embodiments, the first region is configured to slidingly fit within the lumen. In embodiments, a length of the tip member is selected to be greater than a length of the tubular body when the tubular body is in the second expanded state. In embodiments, the distal end of the tubular body defines an opening configured to slide along the tip member during deployment of the soft anchor. In embodiments, the first region is substantially cylindrical. In embodiments, the tubular body is made of one of suture, tape, braid, or mesh. In embodiments, the suture is a size 2 repair suture. In embodiments, the tubular body is hardened by one or more of: pre-stretching, heat stiffening, or coating. In embodiments, a diameter of the first region is selected to be smaller than a diameter of the distally facing surface. In embodiments, the tension is applied to the suture by the tension actuator. In other embodiments, the tension is applied to the suture by hand.

Embodiments of a method of deploying a soft anchor of this disclosure include inserting a soft anchor of a soft anchor deployment system into tissue. The soft anchor deployment system further includes an insertion instrument having a handle with a tensioning actuator at a proximal end and a shaft extending distally from the handle up to a distally facing surface. A tip member extends distally from the distal facing surface and has a first region. The soft anchor includes a tubular body defining a lumen. The tubular body is positioned on the tip member such that the tip member is disposed within and along the lumen. A suture operatively couples to the tubular body and to the tensioning actuator. The method further includes tensioning the suture to deploy the tubular body from a first elongated state to a second expanded state, whereby the soft anchor is set into the tissue, and removing the tip member from the tubular body.

In further embodiments, the method also includes applying additional tension on the suture to remove any resident volume left within the tubular body. In embodiments, tensioning the suture includes tensioning the suture with the tensioning actuator. In other embodiments, tensioning the suture includes tensioning the suture by hand. In embodiments, the tip member has a second region configured to provide slidable clearance for passage of limbs of the suture within the shaft. In embodiments, the second region includes a tab having a wider portion defining flat opposing sides. A diameter of the wider portion is selected to match an inner diameter of the shaft. In embodiments, the method further includes passing the limbs of the suture through the shaft along the flat opposing sides between the tab and the shaft.

A reading of the following detailed description and a review of the associated drawings will make apparent these and other features and advantages. Both the foregoing general description and the following detailed description are explanatory only and are not restrictive of aspects as claimed.

### BRIEF DESCRIPTION OF THE DRAWINGS

The disclosure will be more fully understood by reference to the detailed description, in conjunction with the following figures, wherein:

FIG. 1 illustrates a soft anchor deployment system, according to an embodiment of the disclosure;

FIG. 2 is a detailed view of the shaft and the tip member of the soft anchor deployment system, according to an embodiment of the disclosure;

FIGS. 3A and 3B are detailed views of the soft anchor positioned on the tip member, according to an embodiment of the disclosure;

FIGS. 4A and 4B are detailed views the distal components of the soft anchor deployment system in a disassembled view (FIG. 4A) and an assembled view (FIG. 4B), according to an embodiment of the disclosure;

FIGS. 5A and 5B are detailed views of the soft anchor in an undeployed state (FIG. 5A) and a deployed state (FIG. 5B), according to an embodiment of the disclosure; and

FIG. 6 is a flowchart of a method of deploying a soft anchor using the soft anchor deployment system, according to an embodiment of the disclosure.

### DETAILED DESCRIPTION

In the description that follows, like components have been given the same reference numerals, regardless of whether they are shown in different embodiments. To illustrate embodiment(s) in a clear and concise manner, the drawings may not necessarily be to scale, and certain features may be shown in somewhat schematic form. Features that are described and/or illustrated with respect to one embodiment may be used in the same way or in a similar way in one or more other embodiments and/or in combination with or instead of the features of the other embodiments.

As used in the specification and claims, for the purposes of describing and defining the invention, the term “substantially” is used to represent the inherent degree of uncertainty that may be attributed to any quantitative comparison, value, measurement, or other representation. The term “substantially” is also used herein to represent the degree by which a quantitative representation may vary from a stated reference without resulting in a change in the basic function of the subject matter at issue.

Unless specifically stated or obvious from context, the term “about” is understood as within a range of normal tolerance in the art. Unless otherwise clear from context, all numerical values provided herein are modified by the term “about.” Provided ranges are understood to be shorthand for all the values within the range. “Comprise,” “include,” and/or plural forms of each are open ended and include the listed parts and can include additional parts that are not listed. “And/or” is open-ended and includes one or more of the listed parts and combinations of the listed parts. Use of the relative terms “top,” “bottom,” “above,” “below” and the like helps only in the clear description of the disclosure and does not limit the structure, positioning and/or operation of the disclosure in any manner.

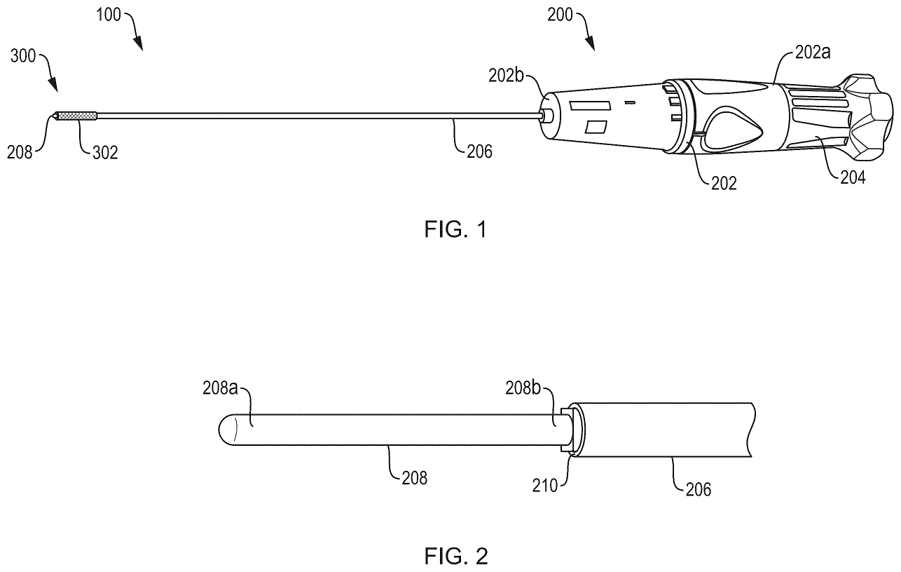

FIG. 1 illustrates a soft anchor deployment system 100, according to an embodiment of the disclosure. As shown in FIG. 1, the soft anchor deployment system 100 may generally include an insertion instrument 200 that has a handle 202 and a tensioning actuator 204 at a proximal end 202a of the handle 202. A hollow shaft 206 may extend from a distal end 202b of the handle 202. A soft anchor 300 may include a tubular body 302 positioned on a tip member 208 that extends distally from the shaft 206. Non-limiting examples of soft anchors 300 of this disclosure are described in U.S. Pat. No. 9,962,149 to ArthroCare Corporation (Austin, TX), incorporated herein by reference. One or more sutures (not shown) may be operatively coupled to the tubular body 302 of the soft anchor 300. For example, the sutures may be woven or stitched through the tubular body 302. The sutures may extend through the shaft 206 and operatively couple to the tensioning actuator 204 such that actuation of the tensioning actuator 204 applies tension on the sutures. Tensioning the sutures may deploy the tubular body 302 from a first, elongated state (as shown) to a second expanded state, as further described below. A length of the tip member 208 may be selected to be greater than a length of the tubular body 302 such that the tip member 208 extends from a distal end of the tubular body 302.

FIG. 2 is a detailed view of the shaft 206 and the tip member 208 of the soft anchor deployment system 100, according to an embodiment of the disclosure. As shown in FIG. 2, the shaft 206 may extend from the handle 202 up to a distally facing surface 210. The tip member 208 may be fixedly coupled to and extend distally from the distally facing surface 210. The tip member 208 may have a first region 208a and a second region 208b. The first region 208a may be substantially cylindrical and may be configured to slidingly fit within the soft anchor 300. However, the disclosure contemplates other suitable cross-sections of the first region 208a that can slidingly fit within the soft anchor 300. A diameter of the first region 208a may be selected to be smaller than a diameter of the distal facing surface 210. An interface between the second region 208b and the shaft 206 may be configured to provide clearance for passage of limbs of the suture within or along the shaft 206 and into the inserter handle 202.

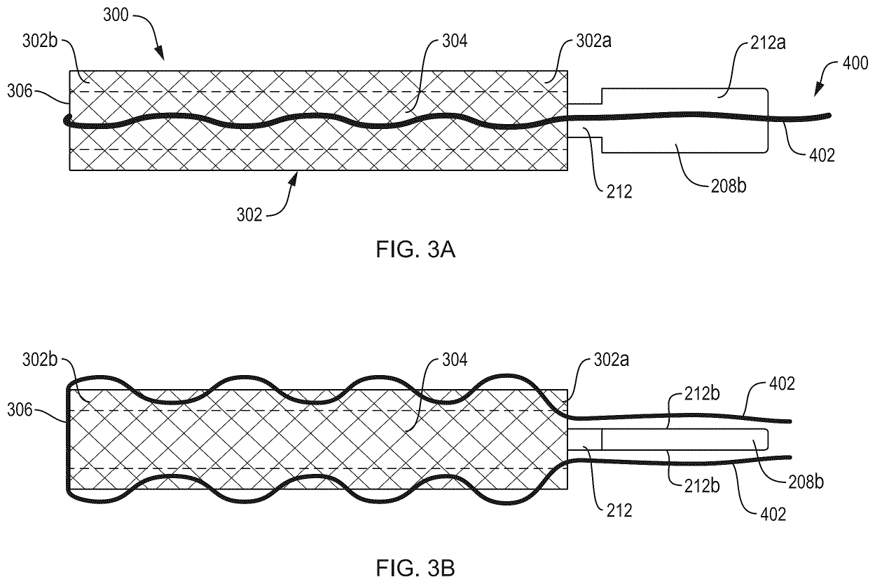

FIGS. 3A and 3B are detailed views of the soft anchor 300 positioned on the tip member 208 in two orthogonal orientations, according to an embodiment of the disclosure. As shown in FIGS. 3A and 3B, the tubular body 302 may define a lumen 304 therethrough that extends from a proximal end 302a to a distal end 302b of the tubular body 302. The tubular body 302 may be made of a soft, flexible material. For example, the tubular body 302 may comprise one of suture, tape, braid, or mesh. In embodiments, the tubular body 302 may be further hardened using standard stiffening techniques, such as pre-stretching, heat stiffening and/or coating with wax or silicone. When the tubular body 302 is positioned on the tip member 208, the first region 208a of the tip member 208 may be disposed within and along the lumen 304. An appropriately sized suture 400 may operatively couple to the tubular body 302 such that limbs 402 of the suture 400 extend proximally from the proximal end 302a of the tubular body 302 along the second region 208b. For example, a #2 size suture may be used for rotator cuff repair or labrum repair in the shoulder, while a #1 size suture may be used with the smaller anchors. However, the disclosure contemplates other suitable suture sizes depending on the type of tissue repair. The distal end 302b of the tubular body 302 may define an opening 306 configured to slide along the tip member 208 during deployment of the soft anchor 300.

Still referring to FIGS. 3A and 3B, the second region 208b may comprise a tab 212 having a wider portion 212a defining flat opposing sides 212b. The wider portion 212a may have a diameter selected to match the inner diameter of the shaft 206, while the flat opposing sides 212b of the tab 212 may provide slidable clearance for the suture limbs 402 between the tab 212 and the shaft 206. While the tab 212 may be configured to provide two suture passages through the shaft 206, the disclosure contemplates other suitable configurations of the tab 212 (such as L-or cross-shaped) that provide more or fewer than two suture passages. The disclosure also contemplates that an inner surface of the shaft 206 could have holes or slots to allow for slidable passage of the suture limbs 402 within the shaft 206.

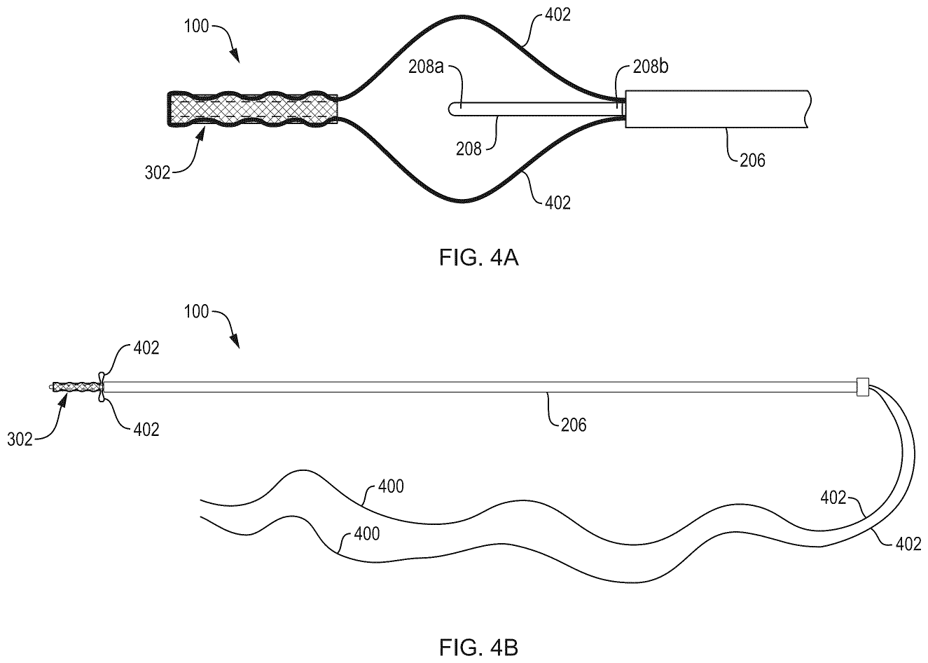

FIGS. 4A and 4B are detailed views the distal components of the soft anchor deployment system 100, according to an embodiment of the disclosure. FIG. 4A illustrates these components in a disassembled view, while FIG. 4B illustrates the components in an assembled view. As shown in FIG. 4A, the limbs 402 of the suture 400 may extend proximally from the tubular body 302 along the second region 208b of the tip member 208 and into the shaft 206. As shown in FIG. 4B, when the tubular body 302 is positioned on the tip member 208 the limbs 402 of the suture 400 may extend through the shaft 206 and into the inserter handle (not shown). In FIG. 4B, the suture limbs 402 are exposed distally from the shaft 206 for ease of illustration, but in actual use the limbs 402 would extend through the handle 202 of the insertion instrument 200.

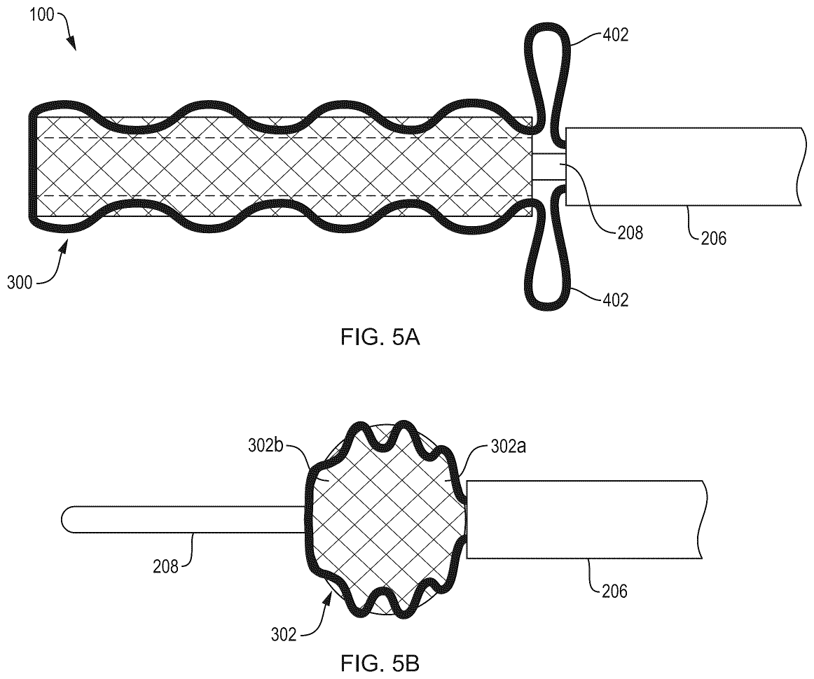

FIGS. 5A and 5B are detailed views of the soft anchor deployment system 100, according to an embodiment of the disclosure. FIG. 5A illustrates soft anchor deployment system 100 when the soft anchor 300 is in an undeployed state, while FIG. 5B illustrates the soft anchor deployment system 100 when the soft anchor 300 is in a deployed state. In FIG. 5A, the suture limbs 402 are shown pulled out laterally from the shaft 206 for ease of illustration, but in actual use the limbs 402 would be pulled into the inner diameter of the shaft 206. As shown in FIG. 5A, the tip member 208 may hold the undeployed soft anchor 300 in its first elongated state for easy insertion into tissue, such as into a bone hole. As shown in FIG. 5B, tension applied to the suture 400 may cause the distal end 302b of the tubular body 302 to slide proximally along and over the tip member 208 and to compress the tubular body 302 against the distally facing surface 210 of the shaft 206 such that the tip member 208 extends distally from the distal end 302b of the tubular body 302. In this deployed state, the soft anchor 300 may be compressed axially and expanded radially relative to its undeployed state to a diameter sufficient to set the soft anchor 300 into bone or other target tissue.

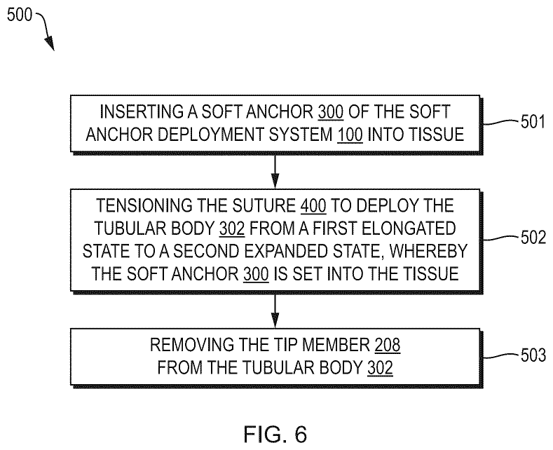

FIG. 6 is a flowchart of a method 500 of deploying a soft anchor 300 using the soft anchor deployment system 100, according to an embodiment of the disclosure. As shown in FIG. 6, Step 501 of the method 500 may include inserting the soft anchor 300 of the soft anchor deployment system 100 into tissue. Step 502 may include tensioning the suture 400 to deploy the tubular body 302 of the soft anchor 300 from a first elongated state to a second expanded state. Step 503 may include removing the tip member 208 from the tubular body 302. Optionally, additional tension may then be applied on the suture 400 to remove any resident volume left within the tubular body 302 after the tip member 208 has been removed. “Resident volume” may be defined as an internal volume that is intentionally formed by the three-dimensional soft anchor 300 in its manufactured, undeployed, or relaxed state. The disclosure also contemplates that the sutures 400 may be tensioned by hand or with the use of some other tensioning mechanism. The method 500 advantageously allows for tissue repair without having to tie knots in the suture 400.

While the disclosure particularly shows and describes preferred examples, those skilled in the art will understand that various changes in form and details may exist without departing from the spirit and scope of the present application as defined by the appended claims. The scope of this present application intends to cover such variations. As such, the foregoing description of examples of the present application does not intend to limit the full scope conveyed by the appended claims.

What is claimed is:

1. A soft anchor deployment system for tissue repair comprising:
an insertion instrument comprising:
a handle having a tensioning actuator at a proximal end;
a shaft extending distally from the handle up to a distally facing surface; and
a tip member extending distally from the distal facing surface, the tip member having a first region;
a soft anchor including a tubular body defining a lumen, the tubular body positioned on the tip member such that the tip member is disposed within and along the lumen; and
a suture operatively coupled to the tubular body and to the tensioning actuator;
wherein tension applied to the suture deploys the tubular body from a first elongated state to a second expanded state; and
wherein, during deployment, a distal end of the tubular body slides along and over the tip member and the tubular body is compressed against the distally facing surface such that the tip member extends distally from a distal end of the tubular body.

2. The soft anchor deployment system of claim 1, wherein the tip member has a second region configured to provide slidable clearance for passage of limbs of the suture within the shaft.

3. The soft anchor deployment system of claim 2, wherein the second region comprises a tab having a wider portion defining flat opposing sides, a diameter of the wider portion selected to match an inner diameter of the shaft.

4. The soft anchor deployment system of claim 3, wherein the flat opposing sides of the tab provide the slidable clearance for the passage of the limbs of the suture between the tab and the shaft.

5. The soft anchor deployment system of claim 1, wherein the first region is configured to slidingly fit within the lumen.

6. The soft anchor deployment system of claim 1, wherein a length of the tip member is selected to be greater than a length of the tubular body when the tubular body is in the second expanded state.

7. The soft anchor deployment system of claim 1, wherein the distal end of the tubular body defines an opening configured to slide along the tip member during deployment of the soft anchor.

8. The soft anchor deployment system of claim 1, wherein the first region is substantially cylindrical.

9. The soft anchor deployment system of claim 1, wherein the tubular body comprises one of suture, tape, braid, or mesh.

10. The soft anchor deployment system of claim 1, wherein the suture is a size 2 repair suture.

11. The soft anchor deployment system of claim 1, wherein the tubular body is hardened by one or more of: pre-stretching, heat stiffening, or coating.

12. The soft anchor deployment system of claim 1, wherein a diameter of the first region is selected to be smaller than a diameter of the distally facing surface.

13. The soft anchor deployment system of claim 1, wherein the tension is applied to the suture by the tension actuator.

14. The soft anchor deployment system of claim 1, wherein the tension is applied to the suture by hand.

15. A method of deploying a soft anchor, comprising:
inserting a soft anchor of a soft anchor deployment system into tissue, the soft anchor deployment system further comprising:
an insertion instrument comprising:
a handle having a tensioning actuator at a proximal end;
a shaft extending distally from the handle up to a distally facing surface; and
a tip member extending distally from the distal facing surface, the tip member having a first region;
the soft anchor including a tubular body defining a lumen, the tubular body positioned on the tip member such that the tip member is disposed within and along the lumen; and
a suture operatively coupled to the tubular body and to the tensioning actuator;
tensioning the suture to deploy the tubular body from a first elongated state to a second expanded state, whereby the soft anchor is set into the tissue; and
removing the tip member from the tubular body.

16. The method of claim 15, further comprising applying additional tension on the suture to remove any resident volume left within the tubular body.

17. The method of claim 15, wherein tensioning the suture comprises tensioning the suture with the tensioning actuator.

18. The method of claim 15, wherein tensioning the suture comprises tensioning the suture by hand.

19. The method of claim 15, wherein the tip member has a second region configured to provide slidable clearance for passage of limbs of the suture within the shaft.

20. The method of claim 19, wherein the second region comprises a tab having a wider portion defining flat opposing sides, a diameter of the wider portion selected to match an inner diameter of the shaft, and wherein the method further comprises passing the limbs of the suture through the shaft along the flat opposing sides between the tab and the shaft.
